(19) United States
(12) Patent Application Publication
(10) Pub. No.: US 2016/0296363 A1
(43) Pub. Date: Oct. 13, 2016

(54) Male Urine Absorption Device

(71) Applicant: Thomas Mc Carthy, Westland, MI (US)

(72) Inventor: Thomas Mc Carthy, Westland, MI (US)

(21) Appl. No.: 14/683,980

(22) Filed: Apr. 10, 2015

(51) Int. Cl. A61F 5/44; A61F 5/453

(52) U.S. Cl. CPC A61F 5/4401; A61F 5/4408; A61F 5/453

(57) ABSTRACT

A male urine absorption device for discretely absorbing urine related to male incontinence includes a sleeve having a first open for receiving a penis inserted into the sleeve and the second open end for extending a head of the penis through the second end such that the sleeve is positioned over and around a shaft of the penis. A extends from the second end of the sleeve. A coupler couples the flap to the sleeve forming a pocket for receiving and enveloping the head of the penis. An absorbing member is positioned in the pocket for absorbing urine discharged from the penis.

### BACKGROUND OF THE DISCLOSURE

### Field of the Disclosure

The disclosure relates to urine absorption devices and more particularly pertains to a new urine absorption device for discretely absorbing urine related to male incontinence.

### SUMMARY OF THE DISCLOSURE

An embodiment of the disclosure meets the needs presented above by generally comprising a sleeve having a first open for receiving a penis inserted into the sleeve and the second open end for extending a head of the penis through the second end such that the sleeve is positioned over and around a shaft of the penis. A extends from the second end of the sleeve. A coupler couples the flap to the sleeve forming a pocket for receiving and enveloping the head of the penis. An absorbing member is positioned in the pocket for absorbing urine discharged from the penis.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top front side perspective view of a male urine absorption device according to an embodiment of the disclosure.

FIG. 2 is a front view of an embodiment of the disclosure.

FIG. 3 is a top front side perspective view of an embodiment of the disclosure in an open position.

FIG. 4 is a cross-sectional view of an embodiment of the disclosure taken along line 4-4 of FIG. 2.

FIG. 5 is a side view of an embodiment of the disclosure in use.

### DESCRIPTION OF THE PREFERRED EMBODIMENT

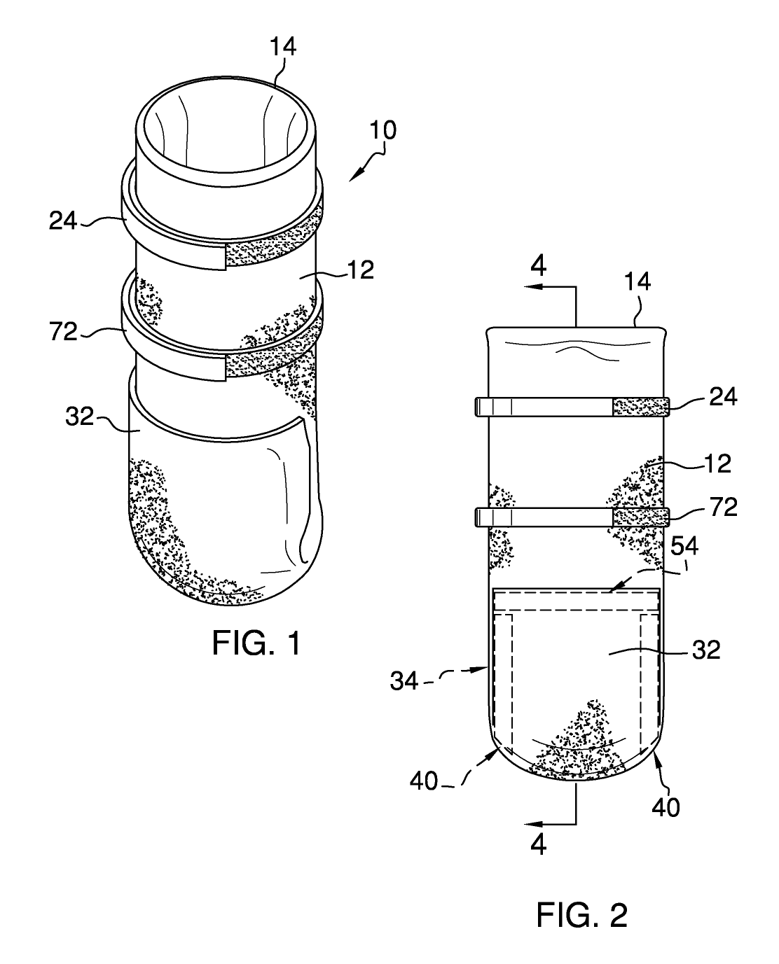

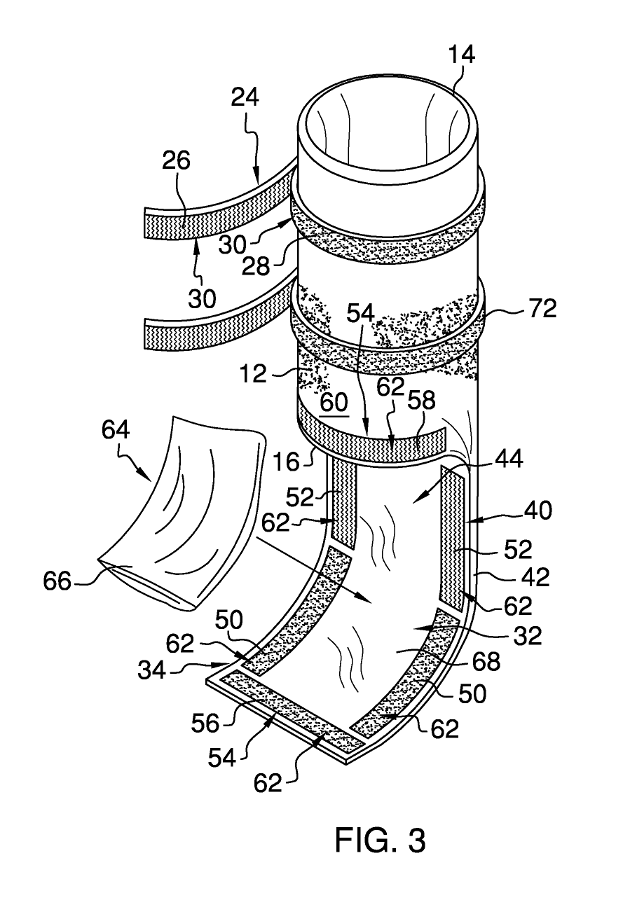

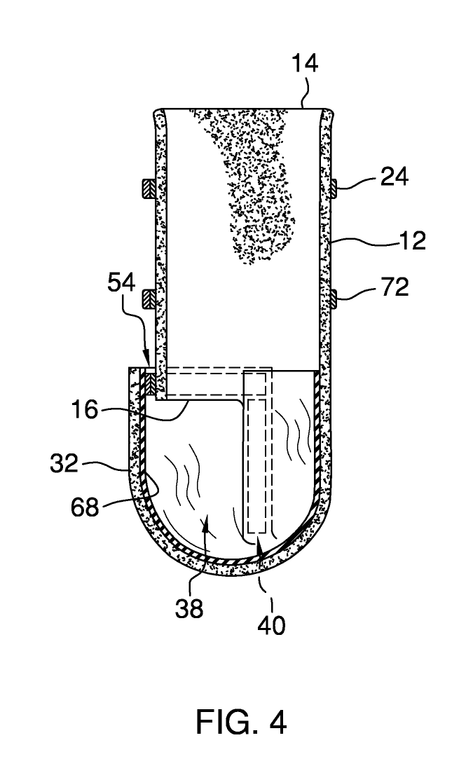

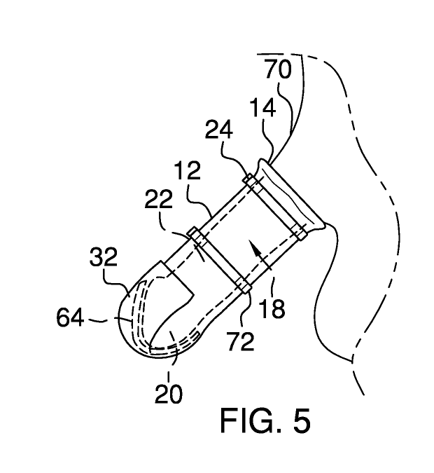

With reference now to the drawings, and in particular to FIGS. 1 through 5 thereof, a new urine absorption device embodying the principles and concepts of an embodiment of the disclosure and generally designated by the reference numeral 10 will be described.

As best illustrated in FIGS. 1 through 5, the male urine absorption device 10 generally comprises a sleeve 12 having a first end 14 and a second end 16. The first end 14 is open for receiving a penis 18 inserted into the sleeve 12 through the first end 14. The second end 16 is open for extending a head 20 of the penis 18 through the second end 16 such that the sleeve 12 is positioned over and around a shaft 22 of the penis 18. A base retaining strap 24 is coupled to the sleeve 12 proximate the first end 14 of the sleeve 12. The base retaining strap 24 is positionable extending around the sleeve 12 such that the base retaining strap 24 secures the sleeve 12 to the shaft 22 of the penis 18 proximate a base 70 of the penis 18. The base retaining strap 24 has opposed faces 26,28 which are covered by complementary portions of hook and loop fastener 30 to secure the base retaining strap 24 in a desired circumference to comfortably retain the sleeve 12 on the penis 18. A medial retaining strap 72 is provided. The medial retaining strap 72 is coupled to the sleeve 12 in spaced relationship to the base retaining strap 24. The medial retaining strap 72 is similarly structured to the base retaining strap 24 to secure the medial retaining strap 72 in a desired circumference to comfortably retain the sleeve 12 on the shaft 22 of the penis 18.

A flap 32 is coupled to the sleeve 12. The flap 32 extends from the second end 16 of the sleeve 12. The flap 32 is integral to the sleeve 12 and the flap 32 and the sleeve 12 are constructed of flannel material, or the like, for comfort. A coupler 34 is coupled to the flap 32 and the sleeve 12 such that a distal end 36 of the flap 32 relative to the sleeve 12 is couplable to the sleeve 12 forming a pocket 38 configured for receiving and enveloping the head 20 of the penis 18. The coupler 34 includes a pair of lateral closures 40. The lateral closures 40 are coupled to and extend along respective lateral sides 42 of a first surface 44 of the flap 32. The first surface 44 of the flap 32 faces the head 20 of the penis 18 when the flap 32 is coupled to the sleeve 12 forming the pocket 38. Each lateral closure 40 includes a distal portion 50 relative to the sleeve 12 and a proximal portion 52 relative to the sleeve 12. The distal portion 50 is engageable to the proximal portion 52. The coupler 34 further includes an end closure 54. The end closure 54 has a first section 56 coupled to the flap 32 and a second section 58 coupled to an outer surface 60 of the sleeve 12. The end closure 54 and each lateral closure 40 are constructed of respective complementary portions of hook and loop fastener 62.

An absorbing member 64 is positioned in the pocket 38 wherein the absorbing member 64 absorbs urine discharged from the penis 18. The absorbing member 64 is a tissue 66 removably positioned within the pocket 38 wherein the absorbing member 64 is disposable and replaceable for reuse of the sleeve 12. A liner 68 is coupled to the flap 32 to define a water resistant barrier around the head 20 of the penis 18. The liner 68 is coextensive with the first surface 44 of the flap 32 and is constructed of a thin plastic sheet material.

In use, the device 10 assists in discretely and comfortably protecting garments from incontinence related to prostrate issues or the like. The penis 18 is inserted through the sleeve 12. The base retaining strap 24 secures the sleeve 12 to the penis 18. The absorbing member 64 is positioned on the first surface 44 of the flap 32 and the flap 32 is secured to form the pocket 38 using the coupler 34.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A male urine absorption device comprising:
a sleeve having a first end and a second end, said first end being open wherein said sleeve is configured for receiving a penis inserted into said sleeve through said first end, said second end being open wherein said second end is configured for extending a head of the penis through said second end such that said sleeve is positioned over and around a shaft of the penis;
a flap coupled to said sleeve, said flap extending from said second end of said sleeve;
a coupler coupled to said flap and said sleeve such that a distal end of said flap relative to said sleeve is couplable to said sleeve forming a pocket configured for receiving and enveloping the head of the penis; and
an absorbing member positioned in said pocket wherein said absorbing member is configured for absorbing urine discharged from the penis.

2. The device of claim 1, further comprising a liner coupled to said flap wherein said pocket is configured to define a water resistant barrier around the head of the penis.

3. The device of claim 2, further comprising said liner being coextensive with a first surface of said flap.

4. The device of claim 1, further comprising said absorbing member being a tissue removably positioned within said pocket wherein said absorbing member is replaceable.

5. The device of claim 1, wherein said coupler comprises a pair of lateral closures, said lateral closures being coupled to and extending along respective lateral sides of a first surface of said flap, each said lateral closure comprising a distal portion relative to said sleeve and a proximal portion relative to said sleeve, said distal portion being engageable to said proximal portion.

6. The device of claim 5, wherein said coupler further comprises an end closure, said end closure having a first section coupled to said flap and a second section coupled to an outer surface of said sleeve.

7. The device of claim 6, further comprising said end closure and each said lateral closure comprising respective complementary portions of hook and loop fastener.

8. The device of claim 1, further comprising a base retaining strap coupled to said sleeve, said base retaining strap being positionable extending around said sleeve such that said base retaining strap is configured for securing said sleeve to the shaft of the penis.

9. The device of claim 1, further comprising said flap extending integrally from said sleeve.

10. The device of claim 9, further comprising said flap and said sleeve being constructed of flannel material.

11. A male urine absorption device comprising:
a sleeve having a first end and a second end, said first end being open wherein said sleeve is configured for receiving a penis inserted into said sleeve through said first end, said second end being open wherein said second end is configured for extending a head of the penis through said second end such that said sleeve is positioned over and around a shaft of the penis;
a base retaining strap coupled to said sleeve, said base retaining strap being positionable extending around said sleeve such that said base retaining strap is configured for securing said sleeve to the shaft of the penis;
a flap coupled to said sleeve, said flap extending from said second end of said sleeve, said flap extending integrally from said sleeve, said flap and said sleeve being constructed of flannel material;
a coupler coupled to said flap and said sleeve such that a distal end of said flap relative to said sleeve is couplable to said sleeve forming a pocket configured for receiving and enveloping the head of the penis, wherein said coupler comprises a pair of lateral closures, said lateral closures being coupled to and extending along respective lateral sides of a first surface of said flap, each said lateral closure comprising a distal portion relative to said sleeve and a proximal portion relative to said sleeve, said distal portion being engageable to said proximal portion, wherein said coupler further comprises an end closure, said end closure having a first section coupled to said flap and a second section coupled to an outer surface of said sleeve, said end closure and each said lateral closure comprising respective complementary portions of hook and loop fastener;
an absorbing member positioned in said pocket wherein said absorbing member is configured for absorbing urine discharged from the penis, said absorbing member being a tissue removably positioned within said pocket wherein said absorbing member is replaceable;
a liner coupled to said flap wherein said pocket is configured to define a water resistant barrier around the head of the penis, said liner being coextensive with a first surface of said flap.
